SEQUENCE LISTING (10 sequences)

SEQ ID NO: 1 - protein, 22 aa, Artificial Sequence
  Synthetic peptide
CEFLVKEVTKLIDNNKTEKEIL

SEQ ID NO: 2 - protein, 18 aa, Artificial Sequence
  Synthetic peptide
YKEVTKLIDNNKTEKEIL

SEQ ID NO: 3 - protein, 12 aa, Artificial Sequence
  Synthetic peptide
LIDNNKTEKEIL

SEQ ID NO: 4 - protein, 8 aa, Artificial Sequence
  Synthetic peptide; all D amino acids
LLEENNDL

SEQ ID NO: 5 - protein, 14 aa, Artificial Sequence
  Synthetic peptide
TXLIDNNATEEILY

SEQ ID NO: 6 - protein, 13 aa, Artificial Sequence
  Synthetic peptide; all D amino acids
YLIEETANNDLAT

SEQ ID NO: 7 - protein, 14 aa, Artificial Sequence
  Synthetic peptide; all D amino acids
YLLEETANNDLLAT

SEQ ID NO: 8 - protein, 11 aa, Artificial Sequence
  Synthetic peptide; all D amino acids
YLLEETANNDL

SEQ ID NO: 9 - protein, 10 aa, Artificial Sequence
  Synthetic peptide; all D amino acids
LLEETANNDL

SEQ ID NO: 10 - protein, 13 aa, Artificial Sequence
  Synthetic peptide
YSLEKETKNNDLL

(19) United States
(12) Patent Application Publication
(10) Pub. No.: US 2002/0128193 A1
(43) Pub. Date: Sep. 12, 2002

(54) Retro-inverso prosaposin-derived peptides and use thereof

(76) Inventors: John S. O'Brien, La Jolla, CA (US); David E. Wright, Ramona, CA (US); Susan L. O'Brien, San Diego, CA (US)

Correspondence Address: KENYON & KENYON, ONE BROADWAY, NEW YORK, NY, 10004, US

(21) Appl. No.: 09/957,143

(22) Filed: Sep. 19, 2001

Related U.S. Application Data
(63) Continuation of application No. PCT/US00/08550, filed on Mar. 29, 2000.
(60) Provisional application No. 60/126,991, filed on Mar. 30, 1999.

(51) Int. Cl. A61K 38/16; C07K 14/00

(52) U.S. Cl. 514/12; 530/324

(57) ABSTRACT

Retro-inverso peptide analogs derived from the active neurotrophic region of saposin C which include the amino acid sequence LLEENNDL (all D-amino acids) (SEQ ID NO: 4). These peptides induce neurite outgrowth in vitro, prevent programmed cell death, induce myelination and have an analgesic effect. They are useful in the treatment of central and peripheral nervous system disorders and neuropathic pain.

### RELATED APPLICATIONS

This application claims the benefit of priority of International Application No. PCT/US00/08550 having international filing date of Mar. 29, 2000, designating the United States of America and published in English, which claims the benefit of priority of U.S. patent application Ser. No. 60/126,991 filed Mar. 30, 1999.

### BACKGROUND OF THE INVENTION

1. Field of the Invention

The present invention relates to neurotrophic peptides. More particularly, the invention relates to retro-inverso neurotrophic peptides derived from prosaposin.

2. Description of the Related Art

Neurotrophins and neurotrophic factors are proteins or peptides capable of affecting the survival, target innervation and/or function of neuronal cell populations (Barde, Neuron 2:1525-1534, 1989). The efficacy of neurotrophins both in vivo and in vitro has been well documented. For example, nerve growth factor (NGF) acts as a trophic factor for forebrain cholinergic, peripheral and sensory neurons (Hefti et al., Neurobiol. Aging 10:515-533, 1989). In vivo experiments indicate that NGF can reverse naturally-occurring as sell as physical traumatic injuries to peripheral nerves (Rich et al., J. Neurocytol. 16:261-268, 1987). Brain-derived neurotrophic factor (BDNF) is a trophic factor for peripheral sensory neurons, dopaminergic neurons of the substantia nigra, central cholinergic neurons and retinal ganglia (Henderson et al., Restor. Neurol. Neurosci., 5:15-28, 1993). BDNF has been shown to prevent normally-occurring cell death both in vitro and in vivo (Hofer et al., Nature 331:261-262, 1988). Ciliary neurotrophic factor (CNTF) promotes survival of chicken embryo ciliary ganglia in vitro and supports survival of cultured sympathetic, sensory and spinal motor neurons (Ip et al., J. Physiol. Paris 85:123-130, 1991).

Prosaposin is the precursor of a group of four small heat-stable glycoproteins which are required for hydrolysis of glycosphingolipids by lysosomal hydrolases (Kishimoto et al., J. Lipid Res. 33:1255-1267, 1992). Prosaposin is proteolytically processed in lysosomes, generating saposins A, B, C and D (O'Brien et al., FASEB J., 5:301-308, 1991). O'Brien et al. (Proc. Natl. Acad. Sci. U.S.A., 91:9593-9596, 1994), U.S. Pat. No. 5,571,787 and published PCT Application No. WO95/03821 disclose that prosaposin and saposin C stimulate neurite outgrowth and promote increased myelination. In addition, U.S. Pat. No. 5,571,787 and PCT WO95/03821 disclose that a 22-mer peptide (CEFLVKEVTKLIDNNKTEKEIL: SEQ ID NO: 1) consisting of amino acids 8-29 of human saposin C stimulates neurite outgrowth in both neuroblastoma cells and mouse cerebellar explants. These references also disclose that an 18-mer peptide (YKEVTKLIDNNKTEKEIL; SEQ ID NO: 2) contained within the active 22-mer of saposin C (with V replaced by Y) also promotes neurite outgrowth and was able to cross the blood brain barrier. O'Brien et al. (FASEB J., 9:681-685, 1995) show that the 22-mer stimulates choline acetyltransferase activity and prevents cell death in neuroblastoma cells in vitro. The active neuritogenic fragment was localized to a linear 12-mer located in the amino-terminal sequence of saposin C (LIDNNKTEKEIL; SEQ ID NO: 3). The 22-mer (SEQ ID NO: 1) is a loop at the adjacent asparagine residues flanked by helical regions in native prosaposin.

A major obstacle to the in vivo therapeutic use of peptides is their susceptibility to proteolytic degradation. Retro-inverso peptides are isomers of linear peptides in which the direction of the sequence is reversed (retro) and the chirality, D or L, of each amino acid is inverted (inverso). There are also partially modified retro-inverso isomers of linear peptides in which only some of the peptide bonds are reversed and the chirality of the amino acid residues in the reversed portion is inverted. The major advantage of such peptides is their enhanced activity in vivo due to improved resistance to proteolytic degradation (For review, see Chorev et al., Trends Biotech. 13:438-445, 1995). Although such retro-inverso analogs exhibit increased metabolic stability, their biological activity is often greatly compromised (Guichard et al., Proc. Natl. Acad. Sci. U.S.A., 91:9765-9769, 1994). For example, Richman et al. (J. Peptide Protein Res. 25:648-662) determined that analogs of linear and cyclic leu-enkephalin modified at the Gly3-Phe4 amide bond had activities ranging from 6-14% of native leu-enkephalin. Chorev et al. (supra.) showed that retro-inversion of a peptide which inhibits binding of vitronectin to its receptor resulted in one peptide which was less potent than the parent isomer by a factor of 50,000, and another peptide which was 4,000 fold more potent than the parent cyclic peptide.

Published International Application No. WO99/12967 discloses retro-inverso peptides derived from the neurotrophic region of saposin C which have between 11 and about 40 amino acids. There is an ongoing need for neurotrophic peptides exhibiting increased metabolic stability while retaining biological activity. The present invention addresses this need.

### SUMMARY OF THE INVENTION

One embodiment of the present invention is a peptide having at least 8 amino acids, and including a peptide having the sequence: D-leu-D-leu-D-glu-D-glu-D-asn-D-asn-D-asp-D-leu (SEQ ID NO: 4). Preferably, the peptide has up to about 40 amino acids. More preferably, the peptide has between 8 and 25 amino acids. Preferably, the peptide has the sequence shown in SEQ ID NO: 4. In one aspect of this preferred embodiment, the peptide is modified at the amino terminus, carboxy terminus, or both amino and carboxy terminus with one of the following independently selected moieties: CH₃CO, CH₃(CH2)ₙCO, C₆H₅CH₂CO and H₂N(CH2)ₙCO, where n=1-10. In another aspect of this preferred embodiment, the peptide is glycosylated at D-asn 5 or at the alphas amino group. Preferably, one or more amide bonds of the peptide is reduced. Advantageously, one or more nitrogens in the peptide is methylated. Preferably, one or more carboxylic acid groups in the peptide is esterified.

The present invention also provides a method for stimulating neuritogenesis or preventing neural cell death, comprising the step of contacting neural cells with a composition comprising an effective neuritogenic or neural cell death-preventing amount of a peptide having at least 8 amino acids, and including the amino acid sequence shown in SEQ ID NO: 4. Preferably, the neuronal cells are neuroblastoma cells.

Another embodiment of the present invention is a method for stimulating myelination or preventing demyelination, comprising the step of contacting neural cells having a myelin sheath with a composition comprising an effective myelination-stimulating or demyelination-inhibiting amount of a peptide having at least 8 amino acids, and including the amino acid sequence shown in SEQ ID NO: 4. Preferably, the peptide has the amino acid sequence shown in SEQ ID NO: 4.

The present invention also provides a method for treating pain in a mammal in need thereof, comprising the step of administering to the mammal a composition comprising an effective myelination-stimulating or demyelination-inhibiting amount of a peptide having at least 8 amino acids, and including the amino acid sequence shown in SEQ ID NO: 4. Preferably, the peptide has the sequence shown in SEQ ID NO: 4. Advantageously, the administering step is intravenous, pulmonary, intrathecal, intramuscular, intradermal, subcutaneous, intracranial, epidural, topical or oral.

Another embodiment of the present invention is a peptide which includes the amino acid sequence shown in SEQ ID NO: 4 for use in stimulating neuritogenesis, preventing neural cell death, stimulating myelination, preventing demyelination and treating neuropathic pain. Preferably, the peptide has up to about 40 amino acids. More preferably, the peptide has between 8 and 25 amino acids. Most preferably, the peptide has between 8 and 15 amino acids. Advantageously, the peptide has the amino acid sequence shown in SEQ ID NO: 4. In one aspect of this preferred embodiment, the peptide is modified at the amino terminus, carboxy terminus, or both amino and carboxy terminus with one of the following independently selected moieties: CH₃CO, CH₃(CH2)ₙCO, C₆H₅CH₂CO and H₂N(CH2)ₙCO, where n=1-10. In another aspect of this preferred embodiment, the peptide is glycosylated at D-asn 5 or at the alpha amino group. Preferably, one or more amide bonds of the peptide is reduced. Advantageously, one or more nitrogens in the peptide is methylated. Preferably, one or more carboxylic acid groups in the peptide is esterified.

The present invention also provides the use of a peptide which includes the amino acid sequence shown in SEQ ID NO: 4 in the preparation of a medicament for stimulating neuritogenesis, preventing neural cell death, stimulating myelination, preventing demyelination and treating neuropathic pain. Preferably, the peptide has up to about 40 amino acids. More preferably, the peptide has between 8 and 25 amino acids. Most preferably, the peptide has between 8 and 15 amino acids. Advantageously, the peptide has the amino acid sequence shown in SEQ ID NO: 4. In one aspect of this preferred embodiment, the peptide is modified at the amino terminus, carboxy terminus, or both amino and carboxy terminus with one of the following independently selected moieties: CH₃CO, CH₃(CH2)ₙCO, C₆H₅CH₂CO and H₂N(CH2)ₙCO, where n=1-10. In another aspect of this preferred embodiment, the peptide is glycosylated at D-asn 5 or at the alpha amino group. Preferably, one or more amide bonds of the peptide is reduced. Advantageously, one or more nitrogens in the peptide is methylated. Preferably, one or more carboxylic acid groups in the peptide is esterified.

### BRIEF DESCRIPTION OF THE DRAWINGS

FIG. 1 is a graph showing the number of spinal cord lesions per mm² in experimental allergic encephalomyelitis (EAE) rats orally administered peptide D8 (100 μg/kg daily) beginning at the onset of EAE (12-14 days after injection of guinea pig spinal cord emulsion and complete Freund's adjuvant).

FIG. 2 is a graph showing the average spinal cord lesion size in experimental allergic encephalomyelitis (EAE) rats orally administered peptide D8 (100 μg/kg daily) beginning at the onset of EAE (12-14 days after injection of guinea pig spinal cord emulsion and complete Freund's adjuvant).

### DETAILED DESCRIPTION OF THE PREFERRED EMBODIMENTS

The present invention provides saposin C-derived retro-inverso (RI) peptide compositions comprising a peptide which includes the amino acid sequence shown in SEQ ID NO: 4. In a preferred embodiment, the peptide has up to about 40 amino acids. In a more preferred embodiment, the peptide has between about 8 and 25 amino acids. The peptide shown in SEQ ID NO: 4 is referred to herein as D8. These retro-inverted (RI) saposin C-derived peptides stimulate neurite outgrowth, prevent neural cell death, stimulate myelination and inhibit demyelination.

Guichard et al. (TIBTECH 14, 1996) teach that retro-inverso (all-D-retro) antigenic mimicry may only occur with peptides in random coil, loop or cyclic conformations. In the case of “helical” peptides, adequate functional mimicry would be expected only if the helicity was, in fact, absent under the solvent conditions used for assessing antigenic mimicry. Thus, the excellent activity of D8, which is believed to adopt an overall helical conformation, is surprising, because it is unlikely that the RI analogs would adopt the same conformation required for binding to the prosaposin receptor as the corresponding all L-native peptide, especially in view of Guichard et al. (TIBTECH, supra.).

Completely or partially RI saposin C-derived peptides having between 8 and about 40 amino acids, preferably between 8 and about 25 amino acids, and more preferably between 8 and about 15 amino acids, and including the amino acid sequence shown in SEQ ID NO: 4, and neurotrophic and/or myelinotrophic analogs thereof, possess significant therapeutic applications in promoting functional recovery after toxic, traumatic, ischemic (e.g. stroke), degenerative and inherited lesions to the peripheral and central nervous system. In addition, these RI peptides stimulate myelination and counteract the effects of demyelinating diseases (i.e. inhibit demyelination). These peptides stimulate the outgrowth of neurons, promote neuroprotection and prevent programmed cell death in neuronal tissues and myelinating glia (i.e. oligodendrocytes) in mammals, preferably humans. The peptides of the invention can also be used to treat various neuropathies including, but not limited to, motor, sensory, peripheral, taxol-induced and diabetic neuropathies. The term “neuropathy” refers to a functional disturbance or pathological change in the peripheral nervous system, and is characterized clinically by sensory or motor neuron abnormalities. The peptides of the invention are also useful as analgesics, particularly for the treatment of neuropathic pain which can develop days or months after a traumatic injury and is often long-lasting or chronic, and in the treatment of sensory and peripheral neuropathy.

One embodiment of the present invention is a method for facilitating neurite outgrowth in differentiated or undifferentiated neural cells by administering to the cells an effective, neurite outgrowth-facilitating amount of a RI saposin C-derived peptide encompassing the RI active 8-mer region shown in SEQ ID NO: 4 or variations thereof as described below.

Variations of these peptide sequences contemplated for use in the present invention include minor insertions and deletions. Conservative amino acid replacements are contemplated. Such replacements are, for example, those that take place within a family of amino acids that are related in the chemical nature of their side chains. The families of amino acids include the basic charged amino acids (lysine, arginine, histidine); the acidic charged amino acids (aspartic acid, glutamic acid); the non-polar amino acids (alanine, valine, leucine, isoleucine, proline, phenylalanine, methionine, tryptophan); the uncharged polar amino acids (glycine, asparagine, glutamine, cysteine, serine, threonine, tyrosine); and the aromatic amino acids (phenylalanine, tryptophan and tyrosine). In particular, it is generally accepted that conservative amino acid replacements consisting of an isolated replacement of a leucine with an isoleucine or valine, or an aspartic acid with a glutamic acid, or a threonine with a serine, or a similar conservative replacement of an amino acid with a structurally related amino acid, will not have a major effect on the properties of the peptide. The ability of any RI saposin C-derived peptide having between 8 and about 40 amino acids, and including the sequence shown in SEQ ID NO: 4, or insertions, deletions or substitutions thereof, to promote neurite outgrowth, myelination, inhibit demyelination; and prevent neural cell death can be determined using the assays in the examples presented below.

Various standard chemical modifications may improve the stability, bioactivity and ability of the peptide to cross the blood brain barrier. One such modification is aliphatic amino terminal modification with a derivative of an aliphatic or aromatic amino acid, forming an amide bond. Such derivatives include, for example, CH₃CO, CH₃(CH₂)CO (n=1-10), C₆H₅CH₂CO, H₂N—(CH₂)ₙCO (n=1-10). Another modification is carboxy terminal modification with a derivative of an aliphatic or aromatic amine/alcohol coupled to the peptide via an amide/ester bond. Such derivatives include those listed above. The peptides may also have both amino and carboxy terminal modifications, wherein the derivatives are independently selected from those listed above. The peptides may also be glycosylated, wherein either the alpha amino group of the D-Asn 5 of the peptide shown in SEQ ID NO: 4, or both, are modified with glucose or galactose. In another contemplated modification, selected backbone amide bonds are reduced (—NH—CH₂). Other modifications include N-methylation of selected nitrogens in the amide bonds and esters in which at least one of the acid groups on the peptide are modified as aromatic or aliphatic esters. Any combination of the above modifications is also contemplated.

The ability of any such peptide to stimulate neurite outgrowth or to prevent neural cell death can easily be determined by one of ordinary skill in the art using the procedures described in Examples 1 and 2 below.

The RI peptides of the invention can be used to promote neurite outgrowth in vitro, ex vivo and in vivo. A typical minimum amount of RI peptide for use in vitro is at least about 0.001 ng/ml. Typically, peptide concentrations in the range of 0.001 ng/ml to about 10 ng/ml are used. Effective amounts for any particular cell or tissue can be determined in accordance with Example 1.

The neural cells can be treated in vitro or ex vivo by directly administering the RI peptides of the invention to the cells. This can be done, for example, by culturing the cells in growth medium suitable for the particular cell type followed by addition of the peptide to the medium. When the cells to be treated are in vivo, typically in a vertebrate, preferably a mammal, the composition can be administered by any conventional mode of administration, including oral, intravenous, intramuscular, pulmonary, intradermal, subcutaneous, intracranial, epidural, intrathecal and topical. Peptide D8 can cross the blood brain barrier as shown in Example 4. This example shows that significant amounts of D8 were present in the brain after oral administration in a rat. These RI peptides persist longer in vivo due to the presence of D peptide bonds.

For treatment of neural disorders, direct intracranial injection or injection into the cerebrospinal fluid may also be used in sufficient quantities to give the desired local concentration of peptide. In both cases, a pharmaceutically acceptable injectable carrier is used. Such carriers include, for example, phosphate buffered saline (PBS) and lactated Ringer's solution. Alternatively, the composition can be administered to peripheral neural tissue by direct local injection or by systemic administration.

The RI peptide compositions of the invention can be packaged and administered in unit dosage form such as an injectable composition or local preparation in a dosage amount equivalent to the daily dosage administered to a patient or as a controlled release composition. A septum sealed vial containing a daily dose of the peptide in either PBS or in lyophilized form is an example of a unit dosage. In a preferred embodiment, daily systemic dosages or the RI peptides of the invention based on the body weight of the vertebrate for treatment of neural diseases or as an analgesic are in the range of from about 0.01 to about 10,000 μg/kg. More preferably, daily systemic dosages are between about 0.1 and 1,000 μg/kg. Most preferably, daily systemic dosages are between about 10 and 100 μg/kg. Daily dosages of locally administered material will be about an order of magnitude less. Oral administration is particularly preferred because of the resistance of the peptides to proteolytic degradation in the gastrointestinal system, and the ability of the peptides to cross the blood brain barrier.

In one preferred embodiment of the invention, the RI peptides are administered locally to neural cells in vivo by implantation thereof. For example, polylactic acid, polygalactic acid, regenerated collagen, multilamellar liposomes, and many other conventional depot formulations comprise bioerodible or biodegradable materials that can be formulated with biologically active neurotrophic peptide compositions. These materials, when implanted, gradually break down and release the active material to the surrounding tissue. Infusion pumps, matrix entrapment systems and transdermal delivery devices are also contemplated. The peptides may also be encapsulated within a polyethylene glycol conformal coating prior to implantation, as described, for example in U.S. Pat. No. 5,529,914.

The RI peptides of the invention may also be enclosed in micelles or liposomes. Liposome encapsulation technology is well known. Liposomes may be targeted to specific tissue, such as neural tissue, through the use of receptors, ligands or antibodies capable of binding the targeted tissue. The preparation of these formulations is well known in the art (Radin et al., Meth. Enzymol. 98:613-618, 1983).

There are currently no available pharmaceuticals able to promote full functional regeneration and restoration or the structural integrity of neural systems. This is particularly true of the central nervous system (CNS). Regeneration of peripheral nerves through the use of saposin C-derived RI peptides having between 8 and about 40 amino acids, and including the sequence shown in SEQ ID NO: 4, is within the scope of the present invention. Moreover, the RI peptides of the invention may be therapeutically useful in the treatment of neurodegenerative diseases associated with the degeneration of neural populations or specific areas of the brain. The principal cause of Parkinson's disease is the degeneration of dopaminergic neurons of the substantia nigra. Since antibodies against prosaposin immunohistochemically stain the dopaminergic neurons of the substantia nigra in human brain sections, the RI peptides of the invention may be therapeutically useful in the treatment of Parkinson's disease. Retinal neuropathy, an ocular neurodegenerative disorder leading to loss of vision in the elderly, is also treatable using the RI peptides of the invention.

It has long been believed that in order to reach neuronal populations in the brain, neurotrophic factors would have to be administered intracerebrally since these proteins do not cross the blood brain barrier. U.S. Pat. No. 5,571,787 discloses that an iodinated neurotrophic 18-mer fragment derived from saposin C crosses the blood brain barrier. Example 4 below shows that an iodinated RI saposin C-derived 8-mer having the amino acid sequence shown in SEQ ID NO: 4 also crosses the blood brain barrier and is found in rat brain in significant amounts after oral administration. It is believed that RI saposin C-derived peptides having up to about 40 amino acids, and including the sequence shown in SEQ ID NO: 4, will also cross the blood brain barrier. Other neuronal populations, including motor neurons, can also be treated by intravenous injection, although direct injection into the cerebrospinal fluid is also envisioned as an alternate route.

Cells may be treated to facilitate myelin formation or to prevent demyelination in the manner described above in vivo, ex vivo or in vitro. Diseases resulting in demyelination of nerve fibers including multiple sclerosis (MS), acute disseminated leukoencephalitis, trauma to brain and/or spinal cord, progressive multifocal leukoencephalitis, metachromatic leukodystrophy, adrenal leukodystrophy and maldevelopment of the white matter in premature infants (periventricular leucomalacia) can be slowed or halted by administration of the neurotrophic peptides of the invention to the cells affected by the disease. The ability of peptide D8 to reverse demyelination in the rat experimental allergic encephalomyelitis (EAE) model is shown in Example 5. EAE is a rat model of human multiple sclerosis (MS) in which demyelination resembles that seen in actively demyelinating human MS lesions (Liu et al., Multiple Sclerosis 1:2-9, 1995).

The compositions of the present invention can be used in vitro as research tools for studying the effects of neurotrophic factors and myelin facilitating materials. However, more practically, they have an immediate use as laboratory reagents and components of cell growth media in order to facilitate growth and maintain neural cells in vitro.

The peptides of the invention were synthesized using an automated solid-phase protocol well known in the art (Fmoc α-amino protection). All peptides were purified by high performance liquid chromatography (HPLC) on a reverse-phase column to an extent greater than 95% prior to use. The identity of peptide D8 (SEQ ID NO: 4) was confirmed by mass spectrometry: MH⁺ (expected)=959; MH⁺ (observed)=959.

The following examples are merely illustrative and are not intended to limit the scope of the present invention.

### EXAMPLE 1

### Stimulation of Neurite Outgrowth

NS20Y neuroblastoma cells were grown in DMEM containing 10% fetal calf serum (FCS). Cells were removed with trypsin and plated in 30 mm petri dishes onto glass coverslips. After 20-24 hours, the medium was replaced with 2 ml DMEM containing 0.5% FCS plus 0, 0.5, 1, 2, 4 or 8 ng/ml of the following effector peptides: D1 (TXLIDNNATEEILY, X=D-alanine, SEQ ID NO: 5), D2 (YLIEETANNDLAT, all D-amino acids; SEQ ID NO: 6), D3 (YLLEETANNDLLAT, all D-amino acids; SEQ ID NO: 7), D4 (YLLEETANNDL, all D-amino acids; SEQ ID NO: 8); D5 (LLEETANNDL, all D-amino acids; SEQ ID NO: 9), D6 (YSLEKETKNNDLL; SEQ ID NO: 10) and D8 (LLEENNDL, all D-amino acids; SEQ ID NO: 4). Cells were cultured for an additional 24 hours, washed with PBS and fixed with Bouin's solution (saturated aqueous picric acid/formalin/acetic acid 15:5:1) for 30 minutes. Fixative was removed with PBS and neurite outgrowth was scored under a phase contrast microscope. Cells exhibiting one or more clearly defined neurites equal to or longer than one cell diameter were scored as positive. At least 200 cells were scored in different portions of each dish to determine the percentage of neurite bearing cells and assays were performed in duplicate.

The results are shown in Table 1. Peptide D8 was by far the most potent of the peptides tested, with an ED50 value (0.01 ng/ml) 50 times lower than the next most potent peptide, D5 (0.2 ng/ml). The ED50 value is defined as the half maximal concentration for maximal neurite outgrowth and neural protection in ng/ml.

TABLE 1

| Peptide | Bioactive? | ED5O (ng/ml) |
|---|---|---|
| D1 | YES | 1.00 |
| D2 | YES | 0.8 |
| D3 | YES | 0.4 |
| D4 | YES | 0.4 |
| D5 | YES | 0.2 |
| D6 | YES | 0.27 |
| D8 | YES | 0.01 |

### EXAMPLE 2

### Prevention of Neural Cell Death

NS20Y cells were plated as described in Example 1 and grown on glass coverslips in 0.5% fetal bovine serum for 2 days in the presence or absence of 8 ng/ml effector peptides. Media was removed and 0.2% trypan blue in PBS was added to each well. Blue-staining dead cells were scored as a percentage of the total on an inverted microscope, counting 400 cells in four areas of each well. The average error of duplicates was ±5%. Similar ED50 values were obtained to those shown in Table 1 (within the standard deviation).

### EXAMPLE 3

### Localization and Integrity of Peptide D8 After Injection

Peptide D8 (SEQ ID NO: 4) was iodinated with ¹²⁵I according to the manufacturer's instructions (Pierce Chemical Co., Rockford, Ill.), and 200 μg/kg in PBS was injected intramuscularly into, or administered orally to, an adult male Sprague-Dawley rat. After 20 min., the rat was anesthetized, perfused with PBS and the organs removed and counted in a gamma counter. Results below give ng/g of D4 in each tissue after conversion of cpm to nanograms (Table 2). All organs studied contained 90% or greater intact D8. The trophic concentration was estimated to be about 0.2 ng/g over 20 min.

TABLE 2

| Organ | D8-20 min (intramuscular) | D8-25 min (oral) |
|---|---|---|
| Brain | 1.00 | 5.62 |
| Spinal Cord | 2.90 | 2.15 |
| Eyes | 13.9 | 4.66 |
| Heart | 9.8 | 3.26 |
| Lung | 43.0 | 526 |
| Kidney 1 | 395 | 201 |
| Kidney 2 | 405 | 217 |
| Spleen | 41.7 | 4.9 |
| Liver | 8.0 | 5.6 |
| Muscle | 19.7 | 3.2 |
| Sciatic Nerve | 36.7 | 3.3 |

### EXAMPLE 4

### Reversal of Demyelination in a Rat Model

Experimental allergic encephalomyelitis (EAE) is a rat model of human multiple sclerosis (MS). In rats, EAE is induced by injecting foreign protein (guinea pig spinal cord) which results in inflammation and demyelination in white matter 11 days later.

EAE was induced in Lewis rats by injection of an emulsion of guinea pig spinal cord and complete Freund's adjuvant (CFA). At day 12-14, when weakness was evident, treatment with D8 (SEQ ID NO: 4) was begun (100 μg/kg orally in PBS via a stomach tube) and continued for 16 days every day. Six rats were injected with vehicle only. The number and size of demyelinating lesions (plaques) in the spinal cord per mm² was scored at day 22.

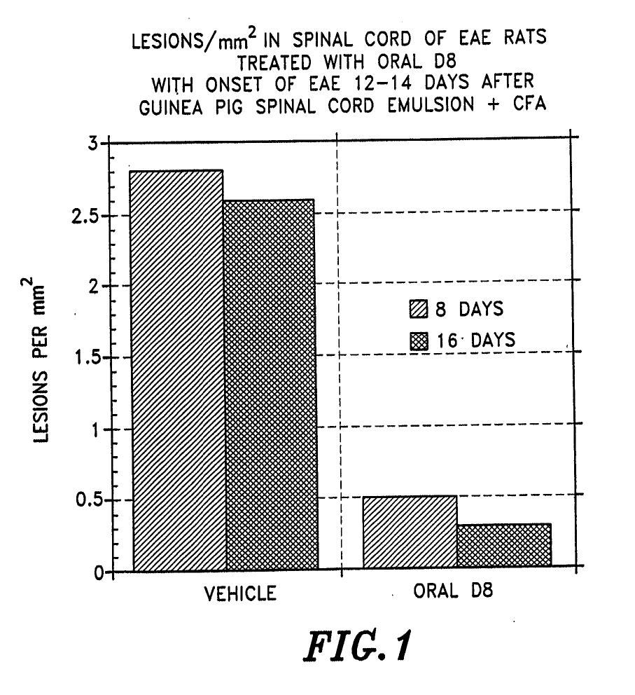

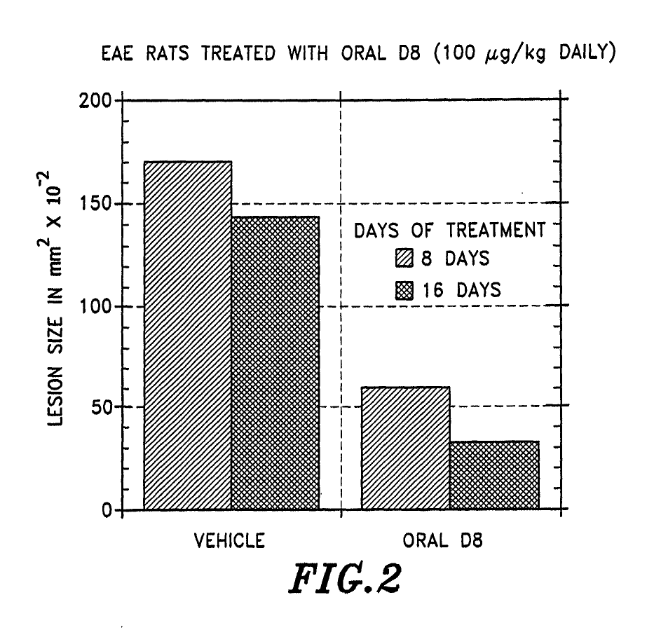

The number of spinal cord lesions is significantly reduced after 8 and 16 days of treatment with D8 compared to control rats injected with vehicle only. After 8 and 16 days of treatment with D8, the number of lesions/mm² was reduced by 76% and 93%, respectively, compared to controls (FIG. 1). In addition. the average lesion size was significantly reduced in D8-treated animals compared to controls. After 8 and 16 days of treatment with D8, the average lesion size was reduced by 65% and 79%, respectively, compared to controls (FIG. 2).

After 16 days of oral treatment with D8 (100 μg/kg/day) beginning after the onset of EAE at 14 days, spinal cord lesions were examined and the number of remyelinated axons per lesion were counted. Animals treated with D8 had lesions which were positive for remyelination as determined by electron microscopy.

There was no difference in weight loss between the control and experimental animals. These results indicate a significant clinical, biochemical and morphological reversal of EAE after systemic treatment with D8. This action differs from the anti-inflammatory effect of current MS drugs which do not act directly upon myelin repair.

### EXAMPLE 5

### Use of RI Peptides in Treating Traumatic Ischemic CNS Lesions

Humans with traumatic lesions to the brain or spinal cord receive systemic injections of about 100 μg/kg peptide D8 or another RI saposin C-derived peptide which includes SEQ ID NO: 4, in a sterile saline solution or in depot form. Improvement is assessed by gain of sensory or motor nerve function (i.e. increased limb movement). Treatments continue until no further improvement occurs.

### EXAMPLE 6

### Use of RI Peptides in Treating Demyelination Disorders

Patients diagnosed with early stage MS are given peptide D8, or a peptide having the amino acid sequence shown in SEQ ID NO: 4, by systemic injection using the same dose range as in Example 8. Dosages are repeated daily or weekly and improvement in muscle strength, musculoskeletal coordination and myelination (as determined by MRI) is observed. Patients with chronic relapsing MS are treated in the same manner when subsequent relapses occur.

### EXAMPLE 7

### Alleviation of Neuropathic Pain in Chung Model Rats

This example describes the effects of bolus intrathecal injection of peptide D8, or another RI saposin C-derived peptide which includes SEQ ID NO: 4, in the Chung experimental model of peripheral neuropathic pain. Each of the four peptides is chemically synthesized, purified, dissolved in sterile PBS and buffered to neutral pH. The surgical procedure previously described by Kim et al. (Pain, 50:355-363, 1992) is performed on male rats to induce an allodynic state. A spinal catheter is introduced two weeks after surgery, Five days later, the peptides are administered at 0.007, 0.07 and 0.7 μg/rat. Pressure thresholds are then determined using calibrated von Frey hairs. The longer the time taken for an animal to withdraw the paw in response to applied pressure, the less severe the neuropathic pain. The peptides significantly increase the threshold pressure, indicating a significant alleviation of neuropathic pain.

### EXAMPLE 8

### Treatment of Sensory Neuropathy

In diabetes, there is an associated sensory neuropathy in which thermal perception is impaired. Streptozoticin-induced diabetic rats are tested for thermal response latency using a Hargraves thermal testing apparatus. Rats are placed on a surface and laser light is shined on a footpad. The response time is then measured in seconds as the time it takes for the rat to withdraw its paw from the surface. Diabetic rats have an increased response time compared to healthy control animals due to the diabetes-induced neuropathy. However, in animals treated with 20, 200 or 1,000 μg/kg of peptide, this response time is significantly reduced. A similar experiment is performed with taxol to induce taxol-mediated neuropathy. Taxol (50 mg/kg) is administered either in the presence or absence of peptide. The rats which receive both taxol and peptide exhibit a decrease in withdrawal time, indicating an improvement in taxol-mediated neuropathy.

It should be noted that the present invention is not limited to only those embodiments described in the Detailed Description. Any embodiment which retains the spirit of the present invention should be considered to be within its scope. However, the invention is only limited by the scope of the following claims.

What is claimed is:

1. A neurotrophic peptide which includes the amino acid sequence shown in SEQ ID NO: 4.

2. The peptide of claim 1, wherein said peptide has up to about 40 amino acids.

3. The peptide of claim 2, wherein said peptide has between 8 and 25 amino acids.

4. The peptide of claim 3, wherein said peptide has between 8 and 15 amino acids.

5. The peptide of claim 1, wherein said peptide has the amino acid sequence shown in SEQ ID NO: 4.

6. The peptide of claim 1, wherein said peptide is modified at the amino terminus, carboxy terminus, or both amino and carboxy terminus with a moiety independently selected from the group consisting of CH₃CO, CH₃(CH₂)ₙCO, C₆H₅CH₂CO and H₂N(CH₂)ₙCO, wherein n=1-10.

7. The peptide of claim 1, wherein said peptide is glycosylated at Asn 5 or at the alpha amino group.

8. The peptide of claim 1, wherein one or more amide bonds is reduced.

9. The peptide of claim 1, wherein one or more nitrogens in said peptide is methylated.

10. The peptide of claim 1, wherein one or more carboxylic acid groups in said peptide is esterified.

11. A method for stimulating neuritogenesis or preventing neural cell death, comprising the step of contacting neural cells with a composition comprising an effective neuritogenic or neural cell death-preventing amount of a peptide which includes the amino acid sequence shown in SEQ ID NO: 4.

12. The method of claim 11, wherein said peptide has the amino acid sequence shown in SEQ ID NO: 4.

13. The method of claim 11, wherein said neuronal cells are neuroblastoma cells.

14. The method of claim 11, wherein said neuroblastoma cells are NS20Y cells.

15. A method for stimulating myelination or preventing demyelination, comprising the step of contacting neural cells with a composition comprising an effective myelin-stimulating or demyelination-inhibiting amount of a peptide which includes the amino acid sequence shown in SEQ ID NO: 4.

16. The method of claim 15, wherein said peptide has the amino acid sequence shown in SEQ ID NO: 4.

17. A method for treating neuropathic pain in a mammal in need thereof, comprising the step of administering to said mammal an effective neuropathic pain-treating amount of a composition comprising a peptide which includes the amino acid sequence shown in SEQ ID NO: 4.

18. The method of claim 17, wherein said peptide has the amino acid sequence shown in SEQ ID NO: 4.

19. The method of claim 17, wherein said administering step is selected from the group consisting of intravenous, pulmonary, intrathecal, intramuscular, intradermal, subcutaneous, intracranial, epidural, topical and oral.

20. A pharmaceutical composition comprising a peptide which includes the sequence shown in SEQ ID NO: 4, in a pharmaceutically acceptable carrier.

21. The composition of claim 20 in a controlled release formulation.

22. The composition of claim 20 in liposomal form.

23. The composition of claim 20 in lyophilized form.

24. The composition of claim 20, in unit dosage form.

25. A method for stimulating myelination or inhibiting demyelination in a mammal in need thereof, comprising the step of administering to said mammal a composition comprising an effective myelin-stimulating or demyelination-inhibiting amount of a peptide which includes the amino acid sequence shown in SEQ ID NO: 4.

26. The method of claim 25, wherein said peptide has the amino acid sequence shown in SEQ ID NO: 4.

27. The method of claim 25, wherein said administering step is selected from the group consisting of intravenous, pulmonary, intrathecal, intramuscular, intradermal, subcutaneous, intracranial, epidural, topical and oral.

28. A peptide which includes the amino acid sequence shown in SEQ ID NO: 4 for use in stimulating neuritogenesis, preventing neural cell death, stimulating myelination, preventing demyelination and treating neuropathic pain.

29. The peptide of claim 28, wherein said peptide has up to about 40 amino acids.

30. The peptide of claim 29, wherein said peptide has between 8 and 25 amino acids.

31. The peptide of claim 30, wherein said peptide has between 8 and 15 amino acids.

32. The peptide of claim 28, wherein said peptide has the amino acid sequence shown in SEQ ID NO: 4.

33. The peptide of claim 28, wherein said peptide is modified at the amino terminus, carboxy terminus, or both amino and carboxy terminus with a moiety independently selected from the group consisting of CH₃CO, CH₃(CH₂)ₙCO, C₆H₅CH₂CO and H₂N(CH₂)ₙCO, wherein n=1-10.

34. The peptide of claim 28, wherein said peptide is glycosylated at Asn 5 or at the alpha amino group.

35. The peptide of claim 28, wherein one or more amide bonds of said peptide is reduced.

36. The peptide of claim 28, wherein one or more nitrogens in said peptide is methylated.

37. The peptide of claim 28, wherein one or more carboxylic acid groups in said peptide is esterified.

38. Use of a peptide which includes the amino acid sequence shown in SEQ ID NO: 4 in the preparation of a medicament for stimulating neuritogenesis, preventing neural cell death, stimulating myelination, preventing demyelination and treating neuropathic pain.

39. The use of claim 38, wherein said peptide has up to about 40 amino acids.

40. The use of claim 39, wherein said peptide has between 8 and 25 amino acids.

41. The use of claim 40, wherein said peptide has between 8 and 15 amino acids.

42. The use of claim 38, wherein said peptide has the amino acid sequence shown in SEQ ID NO: 4.

43. The use of claim 38, wherein said peptide is modified at the amino terminus, carboxy terminus, or both amino and carboxy terminus with a moiety independently selected from the group consisting of CH₃CO, CH₃(CH₂)ₙCO, C₆H₅CH₂CO and H₂N(CH₂)ₙCO, wherein n=1-10.

44. The use of claim 38, wherein said peptide is glycosylated at Asn 5 or at the alpha amino group.

45. The use of claim 38, wherein one or more amide bonds of said peptide is reduced.

46. The use of claim 38, wherein one or more nitrogens in said peptide is methylated.

47. The use of claim 38, wherein one or more carboxylic acid groups in said peptide is esterified.
